(19) United States
(12) Patent Application Publication
(10) Pub. No.: US 2008/0220409 A1
(43) Pub. Date: Sep. 11, 2008

(54) Antigen of Dengue Virus Type 1

(75) Inventors: Han-Chung Wu, Taipei City (TW); Chin-Tarng Lin, Taipei City (TW); Yun-Ching Chen, Taipei City (TW)

Correspondence Address: WPAT, PC;INTELLECTUAL PROPERTY ATTORNEYS, 2030 MAIN STREET, SUITE 1300, IRVINE, CA, 92614, US

(73) Assignee: NATIONAL TAIWAN UNIVERSITY, Taipei City (TW)

(21) Appl. No.: 11/683,817

(22) Filed: Mar. 8, 2007

(51) Int. Cl. C12Q 1/70; C07K 7/08; C12N 7/00

(52) U.S. Cl. 435/5; 435/235.1; 530/327

(57) ABSTRACT

Antigens and B-cell epitopes derived from dengue virus type 1 are provided. The antigens are specifically immunoreactive with sera from individuals infected with dengue virus type 1 but not reactive with sera from healthy individuals and individuals infected with dengue virus type 2. The antigens and epitopes are useful for development of diagnostic kits and reagents, and are useful tools as well in determining whether an individual is infected with dengue virus type 1, and for distinguishing infection from dengue virus type 2.

### BACKGROUND OF THE INVENTION

1. Field of the Invention

The present invention relates to virus antigens, and more particularly to peptide antigens and B-cell epitopes for identifying dengue virus type 1, and distinguishing thereof from dengue virus type 2.

2. The Prior Arts

Dengue fever (DF) and dengue hemorrhagic fever (DHF) are acute febrile diseases, found in the tropics, and transmitted to humans by the mosquito Aedes aegypti and Aedes albopictus. DF, also called as classic dengue fever, is manifested by a sudden onset of fever, with headache, muscle and joint pains, and rashes. There may also be gastritis with some combination of associated abdominal pain, nausea, vomiting or diarrhea. DHF, however, is characterized by higher fever, abnormalities of hemostasis and vascular permeability, and is often fatal. A small proportion of DHF cases leads to dengue shock syndrome (DSS) which has a high mortality rate. Owing to the severe symptoms caused and distinct prognosis compared with classic dengue fever, DHF and DSS are both called as secondary dengue fever as well.

Two-fifths of the world's population is at risk of dengue virus infection, and it is estimated that 50 million people a year are infected therewith. One percent of the infected with this virus will develop DHF. By virtue of the severe effects arisen, governments pay highly attention upon issues concerning early prevention and treatment against dengue fever. However, up to today, no effective strategies and vaccines have been reported capable of preventing the development of DHF/DSS because pathogenic mechanisms of this disease are unclear. For the reason that dengue viruses are causative agents of DF/DHF, detecting precisely or protecting against dengue viruses effectively during initial stage of infection would be a crucial subject, and would lead to effective control of the pathogenicity and mortality rate.

Dengue viruses (DEN), classified in genus Flavivirus, family Flaviviridae, are divided into four serotypes, dengue virus type 1, 2, 3, and 4 (hereinafter referred to as “DEN-1, 2, 3, and 4”), which have very similar genome sequences and envelope (E) protein antigenic properties. Dengue virus is a kind of single positive-strand RNA virus. The RNA thereof, wraped by capsid, membrane (M) and E proteins, codes for capsid, pre-membrane and structural proteins, and 7 kinds of nonstructural (NS) proteins as well.

Therefore, no matter for virus detection or vaccine development, selection of serotype specific epitopes and investigation of neutralizing B-cell epitopes thereof could provide helpful information for development of diagnostic reagents and vaccine design, and better treatment as well.

Researches relating to the antigens of dengue virus are presently emphasized on DEN-2. B-cell epitopes of DEN-2 have been found using overlapping synthetic peptides (PEPSCAN) to analyze anti-sera (Roehrig, J. T., Bolin, R. A. & Kelly, R. G (1998). Virology 246, 317-328). However, the prior approach requires many overlapping synthetic peptides, and it is difficult to identify conformational epitopes and determine the amino acid-binding motif. A variety of DEN-2 antigenic domains have been studied also by antigen fragments using recombinant or enzyme-cleavage proteins (Mason, P. W., Zugel, M. U., Semproni, A. R., Fournier, M. J. & Mason, T. L. (1990). J Gen Virol 71, 2107-2114; Megret, F., Hugnot, J. P., Falconar, A., Gentry, M. K., Morens, D. M., Murray, J. M., Schlesinger, J. J., Wright, P. J., Young, P. & Van Regenmortel, M. H., et al. (1992). Virology 187, 480-491). Even though these methods have identified antigenic domains consisting of 50-200 amino acid residues, they have not been able to specify an exact epitope consisting of only three to eight amino acids. At present, no serotype-specific and neutralizing B-cell epitopes of DEN-1 have been identified by any of abovementioned methods. In addition, the similarity of amino acid sequences between the four serotypes of DEN ranges from 63.2 to 78.7%. High similarity of amino acid sequences between the four serotypes of DEN makes them difficult to distinguish from each other when using antigens obtained from overlapping synthetic peptides, recombinant or enzyme-cleavage antigen fragments.

Comparatively, researches on DEN-1 antigens are less than DEN-2. To date, neither neutralizing epitopes nor epitopes distinguishable between DEN-1 and other DEN strains are disclosed. Thus, generation of serotype-specific mAbs against DEN-1 and investigation of B-cell epitopes thereof would make it possible to identify DEN-1 specific epitopes and to develop epitope-based peptides for the diagnosis of DEN-1 infection. Furthermore, generation of neutralizing mAbs and investigation of B-cell epitopes would help identify neutralizing epitopes and provide information for vaccine design as well.

### SUMMARY OF THE INVENTION

From upon the abovementioned requirement, a primary objective of the present invention is to find out the specific antigens and epitopes of DEN-1, so that it is available to detect and identify DEN-1 and discriminate it from other types of dengue viruses. According to the present invention, neutralizing monoclonal antibodies against DEN-1 were generated by hybridoma cells firstly, and the neutralizing epitopes of the monoclonal antibodies (hereinafter referred to as “mAbs”) were further identified from a phage-displayed random peptide library.

The specific antigens and epitopes obtained according to the present invention would be useful for the development of DEN-1 serotype-specific diagnostic kit and reagents, mAbs, and vaccines, so as to provide better diagnosis and treatment for dengue fever.

Base on above objectives, the neutralizing mAbs, DA6-7 and DA11-13, against envelope (E) proteins of DEN-1 were generated. By immunoblotting assay, these mAbs were found to recognized E protein of DEN-1 only in native gel but not denatured gel. It showed that these antibodies were able to recognize conformational epitopes but not denatured form thereof. The epitopes of these mAbs were further identified from a phage-displayed random peptide library. Immunopositive phage clones reacted specifically with these mAbs and not reacted with normal mouse serum were selected. These phage clones were further analyzed by sequencing to determine the sequences of the peptides displayed. The peptides displayed having an amino acid sequence set forth as SEQ ID NO: 1, 2, 3, 4 or 5 were provided according to the present invention. Three amino acid residues, Serine (S)/Threonine (T)-Serine (S)-Leucine (L)/Isoleucine (I), were highly conserved in all of these immunopositive phage clones. In addition, the phage-displayed consensus sequences could not be found in the sequence of E protein. Consequently, epitopes found according to the embodiment of the present invention are novel.

Through a selection process called phage display biopanning, the phage display technique makes it possible for the rapid identification of linear epitopes and conformational epitopes. Phage-displayed random peptide libraries provide opportunities to map B-cell epitopes and could readily search for disease-specific antigen mimics, and determine cell- and organ-specific peptides. Due to conformational peptide displayed, the specificity of mAbs selected could be increased. The amino acid sequences of SEQ ID NO: 1, 2, 3, 4 and 5 could not be found in the E protein, and the mAbs used for selection only recognize native E protein. Therefore, epitopes (comprising amino acid sequences of SEQ ID NO: 1, 2, 3, 4 or 5) according to the embodiment of the present invention were mimic natural epitopes (mimotopes) or conformational epitopes.

To confirm that the phage-displayed peptide sequences were the B-cell epitopes of neutralizing mAbs, phage-competitive inhibition assays were performed to determine whether the immunopositive phage competed with E proteins for reactivity with mAbs DA6-7 and DA11-13. The binding activity of DA6-7 and DA11-13 to E proteins was inhibited by immunopositive phage clones. The result showed that the phage displayed peptide sequences, SEQ ID NO: 1 and SEQ ID NO: 2 as well as SEQ ID NO: 3, are the B-cell epitopes of neutralizing mAbs DA6-7 and DA11-13, respectively.

Immunopositive phage clones, displayed with a peptide presented as an amino acid sequence of SEQ ID NO: 1 and SEQ ID NO: 2, were reacted with sera collected from DEN-1, DEN-2 patients, and healthy individuals respectively. The phage clones were shown to react specifically with serum collected from DEN-1 patients only, and not react with serum collected from DEN-2 patients and healthy individuals. Thus, the peptide antigens according to the present invention were provided as a diagnostic marker for the detection of DEN-1 infection as well as to distinguish DEN-1 from DEN-2.

The present invention also provide a kit for detecting the presence of antibodies against DEN-1, which comprises: an anti-human IgG antibody coated on a support; at least a phage clone displayed with the peptide selected from the group consisting of SEQ ID NO: 1 and SEQ ID NO: 2; and an anti-phage antibody conjugated with a signal marker. In still another aspect, the present invention includes a method for diagnosing DEN-1 infection, which comprises the steps of: (a) providing an anti-human IgG antibody, and coating the anti-human IgG antibody on a support; (b) providing serum from a test individual, and reacting the serum with the anti-human IgG antibody coated on the support; (c) reacting a phage clone, displayed with the peptide selected from the group consisting of SEQ ID NO: 1 and SEQ ID NO: 2, with the serum; (d) adding an anti-phage antibody conjugated with a signal marker; and (e) examining the anti-phage antibody bound, and determining whether the test individual is infected with DEN-1.

The present invention is further explained in the following embodiment illustration and examples. Those examples below should not, however, be considered to limit the scope of the invention, it is contemplated that modifications will readily occur to those skilled in the art, which modifications will be within the spirit of the invention and the scope of the appended claims.

### BRIEF DESCRIPTION OF THE DRAWINGS

The present invention will be apparent to those skilled in the art by reading the following detailed description of a preferred embodiment thereof, with reference to the attached drawings, in which:

FIG. 1A is a graph showing the plaque reduction neutralization test results of mAbs DA6-7 and DA11-13 against DEN-1;

FIG. 1B is a photograph showing the inhibition of DEN infection by neutralizing mAbs using immunofluorescence assay;

FIG. 2 is a bar graph showing the significant reactivity of phage clones screened by (A) DA6-7, and (B) DA11-13;

FIG. 3 is a bar graph showing the specific reactivity of selected phage clones with neutralizing mAbs (A) DA6-7, (B) DA11-13, and (C) NM-IgG The mAbs were incubated with ten-fold serial diluted phage clones (10⁹, 10⁸, 10⁷, 10⁶ and 0 pfu). The DA6-7-selected phage clone (DA6-7-C4) bound to the DA 6-7 specifically but did not react with the DA11-13 and NM-IgG DA11-13-selected phage clones (DA11-13-C1, C3, C12 and C36) bound to the DA11-13 specifically but did not react with the DA6-7 and NM-IgG;

FIG. 4 is a photograph showing the results of phage-competitive inhibition assay by immunoblot analysis. The reactivity of (A) DA 6-7 with E protein was inhibited by phage clone DA6-7-C4. The reactivity of DA11-13 with E protein was inhibited by phage clones (B) DA11-13-C1 and (C) DA1′-13-C3; and

FIG. 5 is a bar graph showing the results of capture ELISA for serum samples from patients with DEN-1 infection. Serum samples from DEN-1 and DEN-2 patients were detected by (A) DA6-7-captured DEN-1 and (B) DA11-13-captured DEN-1. (C) and (D) show the ELISA reactivity of phage clones with serum samples from DEN patients and NHS (normal human serum) identified by DA6-7-C4 and DA11-13-C1, respectively. The solid line represents cut-off value.

### DETAILED DESCRIPTION OF THE PREFERRED EMBODIMENTS

### Example 1

Generation and Characterization of mAbs Against DEN-1

To screen the epitopes of DEN-1, mAbs against thereof were needed to generate first. According to the present invention, DEN strain used was a local Taiwan strain, DEN-1 766733, isolated from patients with DF. Four prototype dengue viruses, e.g. DEN-1 (Hawaii), DEN-2 (New Guinea C), DEN-3 (H87) and DEN-4 (1241), were also provided. All viral strains were used to infect mosquito C6/36 cells with growth medium containing 50% Mitsumashi and Maramorsch Insect Medium (MMIM; Sigma) plus 50% Dulbecco's modified Eagle's minimal essential medium (DMEM; GIBCO). The DEN-infected C6/36 cells were incubated at 28° C. for 7 to 9 days, and the viruses were harvested from the supernatants by known methods.

Hybridoma cells secreting anti-DEN-1 antibodies were generated according to standard procedure (Kohler, G & Milstein, C. (1975). Continuous cultures of fused cells secreting antibody of predefined specificity. Nature 256, 495-7). Briefly, female BALB/c mice were immunized i.p. with DEN-1 emulsified in Freund's adjuvant (Sigma, St. Louis, Mo.) four times at 3-week intervals. At 4 day after final immunization, the spleen was removed and the cells fused with NSI/1-Ag4-1 myeloma cells using 50% (v/v) polyethylene glycol (GIBCO BRL). Fused cell pellet was re-suspended in DMEM medium supplemented with 15% FBS, hypoxanthine-aminopterin-thymidine (HAT) medium and hybridoma cloning factor (ICN, Aurora, Ohio). Hybridoma colonies were screened for secretion of mAbs that bound DEN-infected C6/36 cells by enzyme-linked immunosorbent assay (ELISA). Selected clones were subcloned by limiting dilution. Ascitic fluids were produced in pristane-primed BALB/c mice. Hybridoma cell lines were grown in DMEM medium with 10% heat-inactivated FBS. MAbs were affinity-purified by protein G sepharose 4B gel, and then the activity and specificity thereof were measured by the following ELISA and Western blot assay.

For ELISA assay, the mAbs purified in the previous step were serially diluted and added to the plates of DEN-infected cells, and incubated at room temperature for 1 hour. The plates were then washed three times with PBST_{0.1} and incubated with horseradish peroxidase (HRP)-conjugated anti-mouse IgG (Jackson ImmunoResearch Laboratories, West Grove, Pa.). The plates were washed five times with PBST_{0.1} and incubated with the peroxidase substrate o-phenylenediamine dihydrochloride (OPD; Sigma). The reaction was stopped with 3 N HCl, and the plates were read using a microplate reader at 490 nm.

Prior to Western blot analysis, antigens of four serotypes of DEN were prepared first. Briefly, C6/36 cells were infected with DEN strains and then lysed in lysis buffer (25 mM Tris/HCl, pH 7.4, 150 mM NaCl and 1% Nonidet-P40) in the presence of protease inhibitors. Cell debris was removed by centrifugation at 3000×g for 10 minutes at 4° C., and the antigens contained in the pellet were recovered.

The recovered antigens were mixed with an equal volume of the native sample buffer (50 mM Tris/HCl pH 6.8, 0.1% bromophenol blue, 10% glycerol) and separated by SDS-polyacrylamide gel electrophoresis (SDS-PAGE). After electrophoresis, the separated antigens were further transferred to a nitrocellulose membrane (Hybond-C Super; Amersham, Little Chalfont, United Kingdom). Nonspecific antibody-binding sites on membranes were blocked with 5% skim milk in PBS and the membranes were then reacted with primary antibodies, the mAbs purified in the previous step. Afterward the HRP-conjugated goat anti-mouse immunoglobulin (Jackson ImmunoResearch Laboratories, West Grove, Pa.) were added and developed with chemiluminescence reagents (ECL; Amersham).

By previous Western blotting and ELISA assay, the specificity of mAbs with four serotypes of DEN was determined. Among various mAbs generated, mAbs DA6-7 and DA11-13 reacted only to DEN-1 and the E proteins thereof (see Table 1). The specificity of these two mAbs recognized viral proteins were further confirmed by 4G2 (ATCC HB197 against E proteins of four serotypes of DENs) and 15F3 (ATCC HB47 against NS1 proteins of DEN-1) mAbs by immunoblotting assay (data not shown). Furthermore, DA6-7 and DA11-13 recognized E protein of DEN-1 only in native gel but not denatured gel by immunoblotting assay (data not shown). This phenomenon confirms that these antibodies were able to recognize conformational epitopes of DEN-1.

TABLE 1
Characterization of mAbs against DEN-1 by
Western blotting, ELISA and PRNT assay

| | Western blotting | | | | ELISA | | PRNT₅₀ (μg/ml) | | | | |
|---|---|---|---|---|---|---|---|---|---|---|---|
| MAb | D1 | D2 | D3 | D4 | D1 | D3 | D1 | D2 | D3 | D4 | Specificity |
| DA6-7 | + | − | − | − | + | − | <1.25 | − | − | − | E |
| DA11-13 | + | − | − | − | + | − | <1.25 | − | − | − | E |

^{a} D1 = DEN-1; D2 = DEN-2; D3 = DEN-3; D4 = DEN-4
^{b} E = envelope proteins

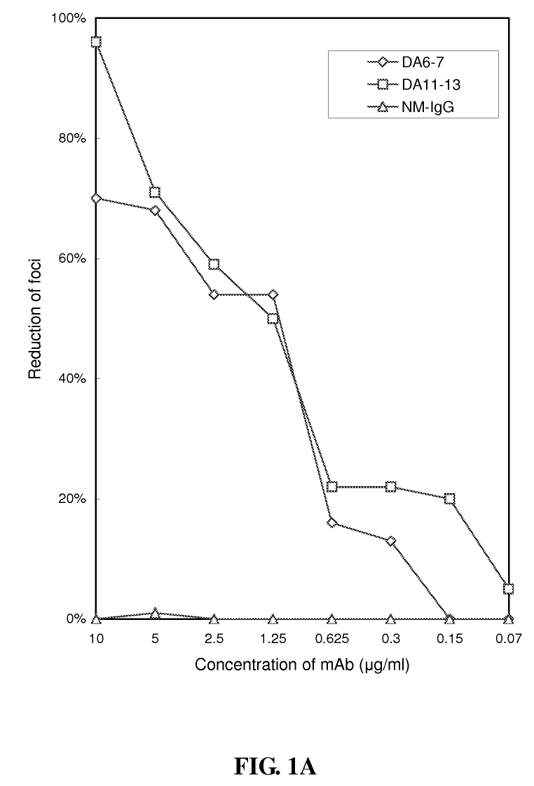

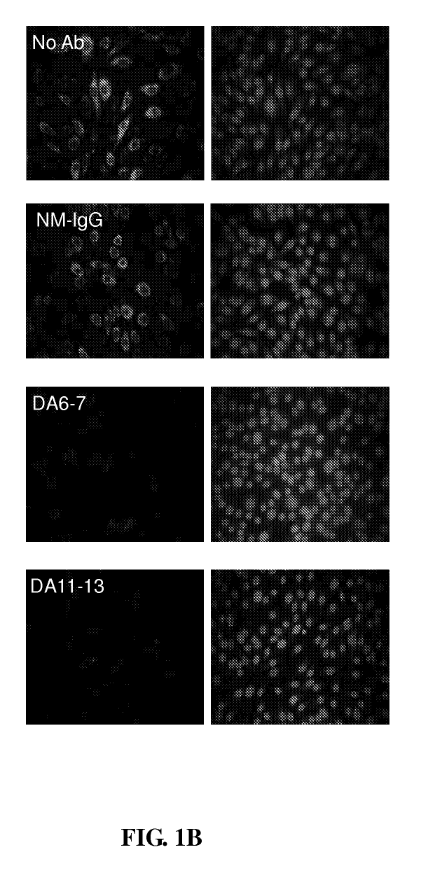

To determine the neutralizing activity against DEN-1 and other DEN stains of DA6-7 and DA11-13, plaque reduction neutralization test (PRNT) was performed. The DA6-7 and DA11-13, as well as normal mouse IgG (NM-IgG) for control, were diluted with serum-free MEM and mixed with an equal volume of various virus suspension and incubated at 37° C. for 1 hour. The antibodies-virus mixture was incubated in duplicate with BHK-21 cells in 12-well plates. After adsorption of viruses for 2 hours, 2 ml of medium (MEM containing 2% fetal bovine serum, antibiotic plus 1% carboxymethyl cellulose) was added to each well. Plates were incubated in 5% CO₂ at 37° C. for 5˜7 days. Cells were then stained with 0.5% crystal violet added directly to the media and left for 60 minutes. After three times washing with tap water, the plaques were counted and the results were shown in Table 1 and FIG. 1A (against DEN-1).

Referring now to FIG. 1A, NM-IgG (10 μg/ml) did not inhibit the formation of plaque, whereas both DA6-7 and DA11-13 had a fifty percent reduction of plaque formation (PRNT₅₀) at 1.25 μg/ml against DEN-1.

In another aspect, to further confirm the neutralizing ability of DA6-7 and DA11-13, immunofluorescence assay was performed. For assay, both mAbs DA6-7 and DA11-13 needed to be incubated with DEN-1 respectively before being used to infect BHK-21 cells. The BHK-21 cells were first seeded in monolayer on sterile glass slides. MAbs DA6-7 and DA11-13 were diluted with serum-free MEM and then mixed with DEN-1 of 0.1 MOI (multiplicity of infection) and incubated at 37° C. for 1 hour. The antibodies-virus mixture was incubated in duplicate with BHK-21 cells. After adsorption of virus for 2 hours, fresh medium (MEM containing 2% fetal bovine serum and antibiotic) was added to each well. Plates were incubated in 5% CO₂ at 37° C. for 2 days. Anti-envelope mAbs 4G2 were then incubated with cells at 4° C. for 1 hour. The glass slides were incubated in a humidified chamber. After three times of washings with PBS, cells were incubated with FITC-conjugated goat anti-mouse IgG. The inhibition of DEN infection in BHK-21 cells by neutralizing mAbs was observed with a fluorescent microscope, and the results were shown in FIG. 1B.

Referring to FIG. 1B, E proteins of DEN-1 were found on BHK-21 cells infected with non-neutralized virions (No antibody and NM-IgG), and the fluorescent staining for dengue E proteins was detected most intensely in the cytoplasm of these cells. In contrast, cells infected by DEN-1 pre-treated with DA6-7 and DA11-13 mAbs showed consistently negative staining. The DEN-1 entry to the BHK-21 cells was inhibited by these neutralizing mAbs and the viruses stopped replicating. Therefore, the fluorescent staining for dengue E proteins was detected faintly in the cytoplasm of these cells.

According to the results of Western blotting, ELISA, PRNT, and immunofluorscence assay, the specificity for DEN-1 of DA6-7 and DA1-13 were confirmed, and both DA6-7 and DA11-13 were also proved to have the neutralizing activity.

### Example 2

Screening immuopositive phage clones with neutralizing antibodies against DEN-1

To identify the B-cell epitopes of DEN-1 specifically recognized by DA6-7 and DA11-13, a phage-displayed random peptide library (New England BioLabs, Inc. Beverly, Mass.) was employed. Through a selection process called biopanning, the phage clones recognized by mAbs DA6-7 and DA11-13 were selected, and the peptide displayed thereby would then be identified.

Before the biopanning step, the ELISA plate was first coated with 100 μg/ml of neutralizing mAbs DA6-7 and DA11-13 respectively in 0.1M sodium bicarbonate buffer (pH 8.6). The plate was incubated with blocking buffer (1% bovine serum albumin in PBS) at 4° C. overnight and then washed with PBST_{0.5} (PBS+0.5% [w/v] Tween-20). The phage displayed 12-mer peptide library was used in the present example, and the phage display biopanning procedures were performed according to known method (Wu, H. C., M. Y. Jung, C. Y. Chiu, T. T. Chao, S. C. Lai, J. T. Jan, and M. E Shaio. 2003. Identification of a dengue virus type 2 (DEN-2) serotype-specific B-cell epitope and detection of DEN-2-immunized animal serum samples using an epitope-based peptide antigen. J Gen Virol 84:2771-2779). Titer of the unamplified third-round phage particles was determined on Luria-Bertani medium-IPTG-X-Gal plates, and immunopositive phage clones selected were then screened by ELISA assay.

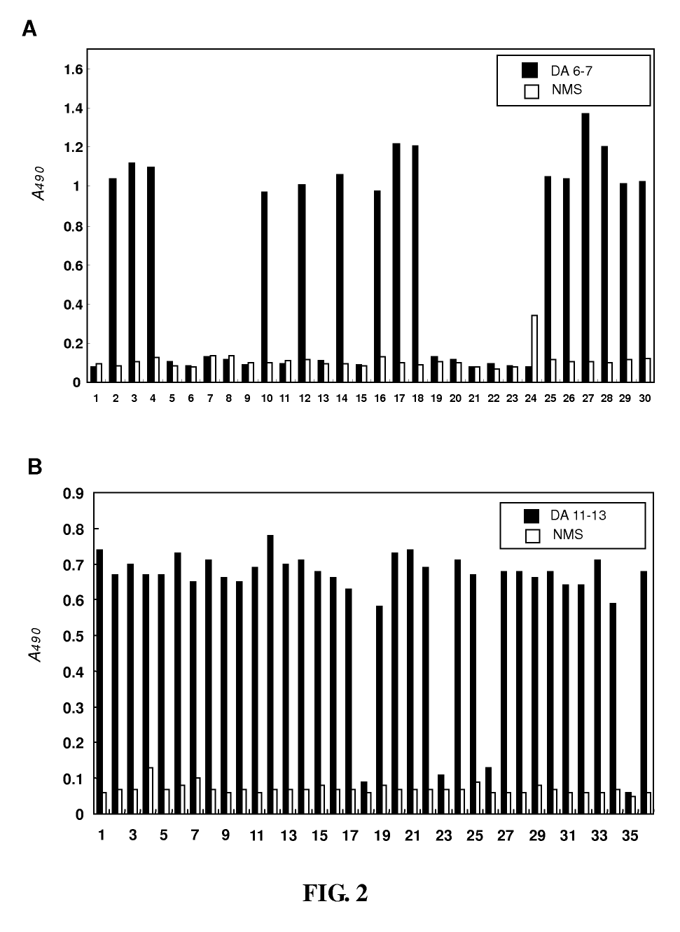

For further identification of phage clones screened, the ELISA plate was coated with 100 μg/ml of mAbs DA6-7 and DA11-13 in 0.1 M sodium bicarbonate buffer (pH 8.6) and blocked with blocking buffer. Serially diluted phages were added to antibody-coated plate and incubated at room temperature for 1 hour. The plate was washed with PBST_{0.5} and then HRP-conjugated anti-M13 antibody (Pharmacia # 27-9411-01) diluted in blocking buffer was added. Afterward the plate was washed five times with PBST_{0.1} and incubated with the peroxidase substrate o-phenylenediamine dihydrochloride (OPD; Sigma). Finally, the reaction was stopped with 3 N HCl. The plate was read using a microplate reader at 490 nm, and the results were shown in FIG. 2.

Referring now to FIG. 2, of the 30 phage clones selected by mAbs DA6-7 (see graph A), fifteen clones (DA6-7-C2, C3, C4, C10, C12, C14, C16, C17, C18, C25, C26, C27, C28, C29 and C30) had significant enhancement of antibody DA6-7 reactivity; they did not bind to normal mouse serum (NMS). Of the 36 phage clones selected by mAbs DA11-13 (see graph B), thirty-two clones (DA11-13-C1, C2, C3, C4, C5, C6, C7, C8, C9, C10, C11, C12, C13, C14, C15, C16, C17, C19, C20, C21, C22, C24, C25, C27, C28, C29, C30, C31, C32, C33, C34 and C36) had significant enhancement of antibody DA11-13 reactivity; they did not bind to NMS.

### Example 3

DNA Sequencing and Sequence Analysis

Immunopositive phage clones were further characterized by DNA sequencing. The phage clones selected from Example 2 were amplified and precipitated with one-sixth volume of polyethylene glycol-NaCl solution (20% (w/v) PEG-8000 and 2.5M NaCl). The precipitated phage pellets were resuspended in 100 μl of iodine buffer (10 mM Tris-HCl, pH 8.0; 1 mM EDTA; 4 M NaI) at room temperature for 10 min after adding 250 μl of ethanol. Phage DNA was isolated from the pellet after centrifugation at 12,000×g for 10 min, washed with 70% ethanol, dried, and resuspended in 50 μl of distilled water. DNA sequences of purified phages were determined according to the dideoxynucleotide chain termination method by automated DNA sequencer (ABI PRISM 377, Perkin-Elmer, Calif., USA). The phage-displayed peptide sequences were translated and aligned with the Genetics Computer Group (GCG) program, and the results were shown in Table 2.

TABLE 2
Alignment of phage-displayed peptide sequences selected by neutralizing mAbs

| | | |
|---|---|---|
| | | SEQ |
| Phage clones | Peptide sequence | ID NO |

DA6-7- selected phage clones

| | | | | | | | | | | | | | |
|---|---|---|---|---|---|---|---|---|---|---|---|---|---|
| DA6-7-C 2, 3, 4, 10, 12, 14, 16, 17, | N | T | Y | F | T | A | F | L | D | G | P | K | 1 |
| 18, 25, 26, 27, 28, 29, 30 | | | | | | | | | | | | | |

DA11-13- selected phage clones

| | | | | | | | | | | | | | |
|---|---|---|---|---|---|---|---|---|---|---|---|---|---|
| DA11-13-C 1, 4, 6, 14, 24, 33 | D | P | L | T | S | L | H | A | M | Q | R | R | 2 |
| DA11-13-C 2, 3 ,8, 11, 13, 20, 28, 30 | Q | V | P | S | S | L | S | L | L | Q | S | R | 3 |
| DA11-13-C 12 | T | A | P | S | S | I | S | L | I | H | A | R | 4 |
| DA11-13-C 5, 36 | H | K | Y | S | S | L | D | L | L | Q | Q | R | 5 |

^{a} Page-displayed consensus amino acids are shown in bold.

Referring now to Table 2, fifteen immunopositive phage clones (DA6-7-C2, C3, C4, C10, C12, C14, C16, C17, C18, C25, C26, C27, C28, C29 and C30) that were highly reactive with DA6-7 were amplified and DNAs thereof were isolated for DNA sequencing. All of the phage clones displayed 12 amino acid residues NTYFTAFLDGPK (SEQ ID NO: 1). Similarly, seventeen immunopositive phage clones (DA11-13-CT, C2, C3, C4, C5, C6, C8, C11, C12, C13, C14, C20, C24, C28, C30, C33 and C36) that were highly reactive with DA11-13 were amplified and DNAs thereof were isolated for DNA sequencing. These immunopositive phage clones displayed four different peptide sequences. Phage clones DA11-13-CT, C4, C6, C14, C24 and C33 displayed DPLTSLHAMQRR (SEQ ID NO: 2). Phage clones DA1′-13-C2, C3, C8, C11, C13, C20, C28 and C30 displayed the same amino acid sequence, QVPSSLSLLQSR (SEQ ID NO: 3), DA11-13-C5 and C36 HKYSSLDLLQQR (SEQ ID NO: 5), and DA11-13-C12 TAPSSISLIHAR (SEQ ID NO: 4). Three amino acid residues, Serine (S)/Threonine (T)-Serine (S)-Leucine (L)/Isoleucine (I), were highly conserved in all of these immunopositive phage clones. However, in this example, the phage-displayed consensus sequences for both neutralizing mAbs described above could not be found in the sequence of E protein of DEN-1. In these cases, epitopes found according to the embodiment of the present invention are novel, and they are mimic natural epitopes (mimotopes) or conformational epitopes.

### Example 4

Identification of Binding Specificity of Mabs and Peptides Displayed on Phage Clones

To confirm that the peptides displayed on immunopositive phage clones bound DA6-7 and DA11-13 specifically, serial dilution of peptide-displayed phage clones (10⁹, 10⁸, 10⁷, 10⁶, and 0 pfu) for ELISA binding assay was performed.

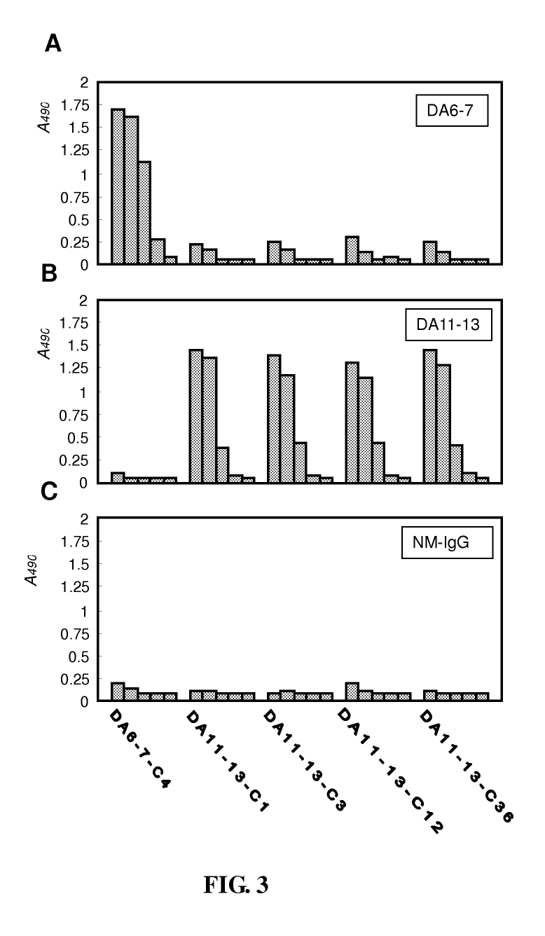

Referring now to FIG. 3, the DA6-7-selected phage clone, DA6-7-C4 (displayed with a peptide having an amino sequence comprising SEQ ID NO: 1), reacted with DA 6-7 specifically and dose dependently but not with DA11-13 and NM-IgG. DA11-13-selected phage clones, DA11-13-C1 (displayed SEQ ID NO: 2), DA11-13-C3 (displayed SEQ ID NO: 3), DA11-13-C12 (displayed SEQ ID NO: 4) and DA11-13-C36 (displayed SEQ ID NO: 5), reacted with DA11-13 specifically and dose dependently but not with DA6-7 and NM-IgG.

### Example 5

Determination of Epitopes on Peptide-Displayed Phage Clones

To further confirm that the phage-displayed peptide sequences were the B-cell epitopes of neutralizing mAbs, phage-competitive inhibition assays were performed to determine whether the immunopositive phage competed with E proteins for reactivity with DA6-7 and DA11-13.

E proteins of DEN-1 mixed with an equal volume of the native sample buffer were separated by SDS-PAGE under denaturing conditions, and then transferred to a nitrocellulose membrane. Nonspecific antibody-binding sites were blocked with 5% skim milk in PBS. MAbs DA6-7 and DA11-13 were diluted with 5% skim milk in PBS (final concentration of 0.2 μg/ml and 7 μg/ml respectively) and mixed with ten-fold serial diluted phage clones (e.g. DA6-7-C4, DA11-13-CT and DA11-13-C3) respectively and incubated at 4° C. for 1 hour. The membranes were reacted with mAbs and phage mixture at 4° C. for 1 hour. HRP-conjugated goat anti-mouse immunoglobulin was added and the membranes were then developed with chemiluminescence reagents.

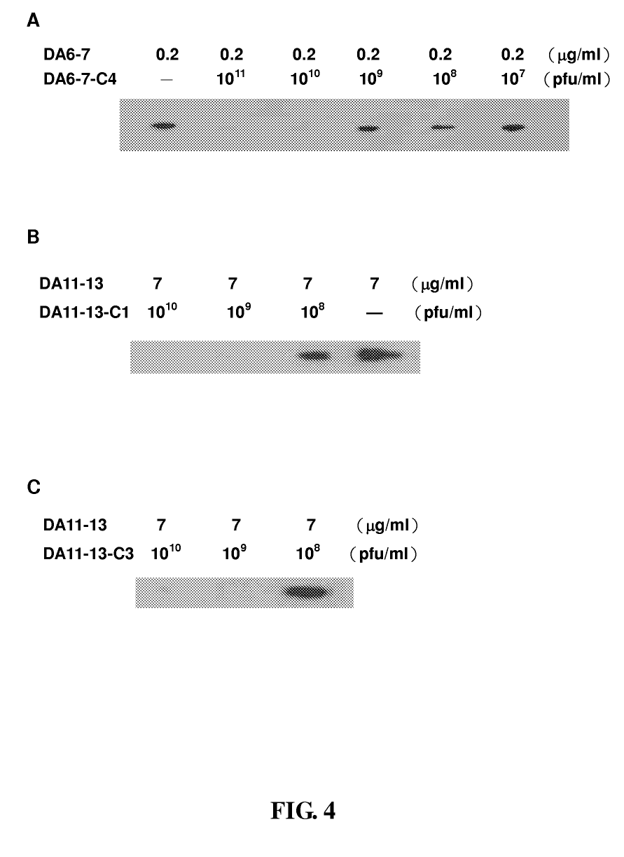

Referring now to FIG. 4, the reaction activity of DA6-7 with E proteins was inhibited markedly by DA6-7-C4 (graph A) at 10¹¹ and 10¹⁰ pfu/ml of phage. Similarly, the reaction activity of DA11-13 with E proteins was inhibited completely by DA11-13-CT (graph B) and DA11-13-C3 (graph C) at 10⁹ and 10¹⁰ pfu/ml of phage, respectively. The binding activity of these neutralizing mAbs to E proteins was exactly inhibited by immunopositive phage clones. These findings strongly support that the phage displayed peptides with sequences of SEQ ID NO: 1 and SEQ ID NO: 2, SEQ ID NO: 3 are indeed the B-cell epitopes of DA6-7 and DA11-13, respectively.

In addition, as a result of having conserved amino sequence, Serine (S)/Threonine (T)-Serine (S)-Leucine (L)/Isoleucine (I), as in SEQ ID NO: 2 and SEQ ID NO: 3, the peptide having the amino acid sequence of SEQ ID NO: 4 and SEQ ID NO: 5 were also be inferred as the B-cell epitopes of DA11-13.

### Example 6

Detection of IgG Antibodies in Serum Samples from Den Patients

To evaluate whether the mAbs DA6-7 and DA11-13 generated according to the present invention could be used for capture antibodies to serological detection of DEN patients, ELISA assay was performed. For assay, ELISA plates were coated with DA6-7 and DA11-13 as capture mAbs for proceeding detection.

Plates were coated with 10 μg/ml of DA6-7 or DA11-13 in 0.1 M sodium bicarbonate buffer (pH 8.6). After incubation at room temperature for 2 hours, the plates were washed with phosphate-buffered saline (PBS; pH 7.2) and blocked with blocking buffer (1% bovine serum albumin in PBS). After blocking, plates were washed and then incubated with diluted DEN-1 virus. After incubation, plates were washed with PBST_{0.1}, and then incubated with 1:200-diluted DEN-1 patient serum or normal human serum (NHS) in blocking buffer. Afterward plates were washed and incubated with HRP-conjugated goat anti-human IgG (Jackson ImmunoResearch Labs, West Grove, Pa.). Plates then underwent the same ELISA procedures as described in the example 2. Mean optical density of NHS at 490 nm (A₄₉₀ nm) plus three times the standard deviation was used to determine the cut-off value for this assay.

Further, to evaluate whether the epitopes of DA6-7 and DA11-13 according to the present invention could be used for serological detection with DEN patients, or whether the epitopes of DA6-7 and DA11-13 could be used to distinguish DEN-1 from DEN-2, ELISA procedures similar to foregoing described was performed. First, ELISA plates were coated with 10 μg/ml purified anti-human IgG capture antibodies (Jackson ImmunoResearch Labs, West Grove, Pa.), blocked with blocking solution and then incubated with the 1:200 diluted tested serum samples. 10⁹ immunopositive phage particles were added to antibody-coated plate and the same procedures as described in the example 2 were followed. Mean optical density of NHS at 490 nm (A₄₉₀ nm) plus three times the standard deviation was used to determine the cut-off value for this assay.

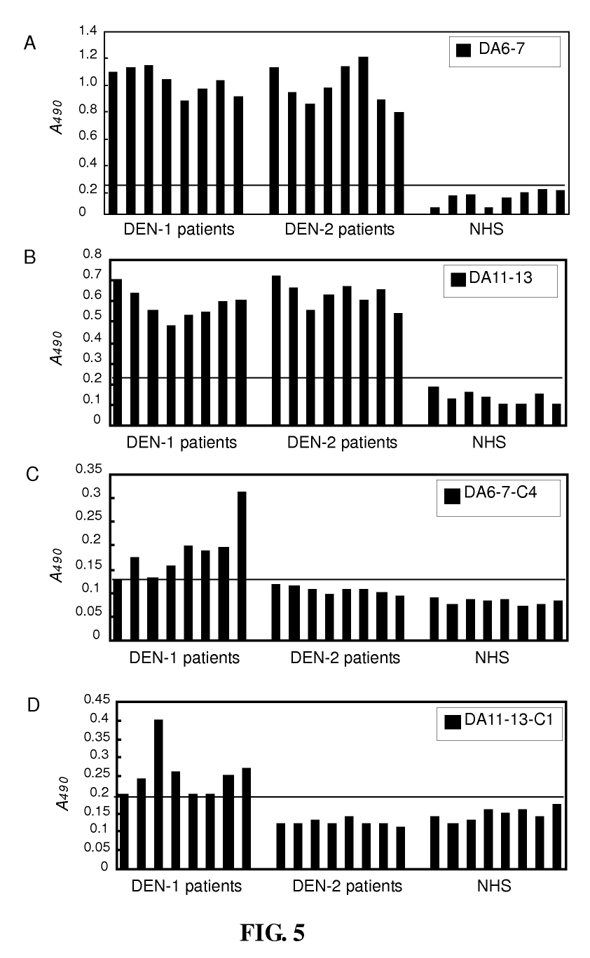

Referring now to FIG. 5, the mAbs-coated plates were bound with DEN-1 and incubated with eight serum samples each of DEN-1 and DEN-2 patients and eight samples of NHS from healthy control subjects. All of the DEN-1 and DEN-2 patients were detected by DA6-7-captured DEN-1 (graph A). Similarly, all of DEN-1 and DEN-2 patients were also detected by DA11-13-captured DEN-1 (graph B). However, all of the control NHS responses to the two mAbs were seronegative.

Whether the phage-displayed epitope could be used as a diagnostic tool to detect antibodies present in serum samples from DEN patients was evaluated according to the embodiment of the present invent. Six of eight serum samples from the DEN-1 patients were seropositive with DA6-7-C4 (graph C). Five of eight serum samples from DEN-1 patients were seropositive with DA11-13-C1 (graph D). None of the control NHS had positive responses with these two phage clones. To further evaluate whether DA6-7-C4 and DA11-13-C1 could be used in the differentiation of serotypes of DEN infection, same method to detect antibodies in DEN-2 patient serum samples was also used. All serum samples obtained from DEN-2 patients were seronegative, indicating that serum antibodies from DEN-1 patients could be detected by phage-displayed epitopes according to the embodiment of the present invent.

Although the present invention has been described with reference to the preferred embodiment thereof, it is apparent to those skilled in the art that a variety of modifications and changes may be made without departing from the scope of the present invention which is intended to be defined by the appended claims.

What is claimed is:

1. An antigen of dengue virus type 1, comprising:
a peptide having an amino sequence selected from the group consisting of SEQ ID NO: 1 and SEQ ID NO: 2.

2. The antigen of dengue virus type 1 as claimed in claim 1, wherein the peptide set forth as an amino sequence of SEQ ID NO: 1 or SEQ ID NO: 2 is a B-cell epitope.

3. The antigen of dengue virus type 1 as claimed in claim 1, wherein the peptide is further displayed on the surface of a phage.

4. The antigen of dengue virus type 1 as claimed in claim 1, wherein the peptide is for development of diagnostic reagents.

5. The antigen of dengue virus type 1 as claimed in claim 1, wherein the peptide shows an immunopositive reaction binding specifically to antibodies in serum from patients of dengue virus type 1 but a negative reaction to antibodies in serum from healthy individuals or patients infected with dengue virus type 2.

6. The antigen of dengue virus type 1 as claimed in claim 5, wherein the peptide set forth as an amino sequence of SEQ ID NO: 1 or SEQ ID NO: 2 is a B-cell epitope.

7. The antigen of dengue virus type 1 as claimed in claim 5, wherein the peptide is further displayed on the surface of a phage.

8. The antigen of dengue virus type 1 as claimed in claim 5, wherein the peptide is for development of diagnostic reagents.

9. A method for diagnosing dengue virus type 1 infection, comprising the steps of:
(a) providing an anti-human IgG antibody, and coating the anti-human IgG antibody on a support;
(b) providing serum from a test individual, and reacting the serum with the anti-human IgG antibody coated on the support;
(c) reacting a phage clone, displayed with the peptide selected from the group of claim 1, with the serum;
(d) adding an anti-phage antibody conjugated with a signal marker; and
(e) examining the anti-phage antibody bound, and determining whether the test individual is infected with dengue virus type 1.

10. An antigen of dengue virus type 1, comprising:
a peptide having an amino sequence selected from the group consisting of SEQ ID NO: 3, SEQ ID NO: 4 and SEQ ID NO: 5.

11. The antigen of dengue virus type 1 as claimed in claim 10, wherein the peptide is set forth as an amino sequence of SEQ ID NO: 3, SEQ ID NO: 4 or SEQ ID NO: 5.

12. The antigen of dengue virus type 1 as claimed in claim 11, wherein the peptide set forth as the amino sequence of SEQ ID NO: 3 is a B-cell epitope.
